(19) United States
(12) Patent Application Publication
(10) Pub. No.: US 2024/0314916 A1
(43) Pub. Date: Sep. 19, 2024

(54) FREE RADICAL GENERATION DEVICE AND METHODS THEREOF

(71) Applicant: SOMNIO GLOBAL HOLDINGS, LLC, Farmington Hills, MI (US)

(72) Inventors: Pravansu S. Mohanty, Canton, MI (US); Volodymyr Ivanovich Golota, Novi, MI (US); Tejasvi Chunduri, Ann Arbor, MI (US); Ramcharan Visveswaran, Westland, MI (US)

(21) Appl. No.: 18/562,517

(22) PCT Filed: May 24, 2022
(86) PCT No.: PCT/US2022/030642

§ 371 Date: Nov. 20, 2023

Related U.S. Application Data
(60) Provisional application No. 63/192,275, filed on May 24, 2021.

(51) Int. Cl. H05H 1/24; H05H 1/52

(52) U.S. Cl. CPC H05H 1/247; H05H 1/52; H05H 2245/15; H05H 2245/20

(57) ABSTRACT

A free radical generator includes a discharge electrode assembly with discharge electrode pins in a radial pattern and electrically configured to receive one or more voltage pulses. A liquid or solid counter electrode has a surface separated from the pins by a discharge gap. A motive element rotates the electrode assembly such that the array of pins rotates about an axis and the pins move relative to the counter electrode. A liquid treatment system may include a free radical generator and introduce free radicals into the liquid. A gas treatment system may include the free radical generator, a gas flow generating element to. a column for the flow of gas. and a liquid introduction element operable to introduce a liquid from a liquid bath into the interior space of the column such that the flow of gas is exposed to the liquid. Methods and further systems are also provided.

### CROSS-REFERENCE TO RELATED APPLICATIONS

This application claims priority to U.S. Provisional App. No. 63/192,275, filed May 24, 2021, all of which is hereby incorporated by reference in its entirety.

### FIELD OF THE INVENTION

The present disclosure relates to a device and method for generating free radicals between a discharge electrode assembly and a counter electrode, which may be liquid in a liquid bath.

### BACKGROUND OF THE INVENTION

Cold plasma discharge through air or molecular gases and the resulting radicals such as: O*, N*, OH*, H*, CH₂*, etc., have many practical applications ranging from sterilization, surface treatment, and pollutant removal from gas streams.

The most widely used method of generating discharge in a gas is by the use of a dielectric barrier between two conductors and then applying a high voltage between the electrodes to cause discharge in the gap, which is commonly known as “DBD discharge” in the art. At a sufficiently high voltage between the electrodes the discharge starts in the gas volume. It spreads out until it reaches the electrodes, but at the dielectric surface it builds up a space charge that cancels the applied electric field. At that moment the discharge stops.

An alternative method utilizes an asymmetric electrode pair without the use of a dielectric barrier between them. Streamer or filament type discharges initiate from regions with strong electric fields that exist at a surface of an electrode with high curvature (needle, wire etc.). After being formed, a streamer is able to propagate for a long distance even in space where the field is relatively weak. However, to prevent complete breakdown (arcing) in the discharge gap, high voltage short pulses are used to stop the discharge before it transitions into an arc. The most common electrodes used in practice for this purpose are point-to-plane and wire-in-cylinder geometries.

Streamer discharge devices utilizing an asymmetric electrode pair are easier to scale up particularly for large gas flows. Typically, the electric field required to ignite streamers in air-like mixtures is about 10²-10³ kV·cm⁻¹. Such high fields can easily be generated by using sharp electrodes such as wire and pins, with a modest applied voltage that is orders of magnitude lower. The field enhancement in front of the streamer head is high enough to ensure a positive net ionization coefficient. A streamer can be considered as a self-sustained ionization wave propagating in neutral gas that is converted into low-temperature plasma behind the wave front, resulting in a channel-like appearance. The interior of the streamer channel consists of a conducting plasma with roughly the same electron and ion densities. The self-induced electric field of the streamer head allows the streamer to continue propagating even into regions where the applied electric field is insufficient to ensure a positive net ionization, and hence gives the scalability to these devices.

One of the problems of electric discharge systems is the stability and lifetime of the electrodes. Electrodes are exposed to electron and ion bombardment, UV irradiation, deposition of reactive particles and electrolytic corrosion. As a result, the original shape of the electrode changes, the electrode surface becomes dirty, and the surface conductivity changes. Also, contamination reduces the emission of electrons, making the emission of electrons uneven over the surface of the electrode. This increases the concentration of current at some small points on the surface of the electrodes and assists to the formation of a spark.

For some applications, it is desirable to provide free radicals in a liquid, for treatment of the liquid or use of the liquid for other purposes. In one approach, free radicals are generated in a gas, such as air, as discussed above and the gas is then added to the liquid to transfer free radicals. However, many of the free radicals are short lived and free radicals are not efficiently added to the liquid in this way.

Another approach has been to use a liquid as the counter electrode. Use of a liquid as the counter electrode addresses some of the above issues. FIG. 1 schematically illustrates such an approach, wherein a discharge electrode 10 is disposed above a liquid bath 12 with a discharge gap 14 between the surface 16 of the liquid 18 in the liquid bath 12 and the tip of the electrode 10. The liquid in the liquid bath is grounded, such as by a ground 20 in the liquid bath 12, such that the liquid itself serves as a counter electrode. Electrical pulses are applied between the discharge electrode 10 and the counter electrode (liquid 18) thereby generating plasma streamer branches 22, as also represented in FIG. 1. This generates free radicals both in the discharge gap 14 and into the liquid 18. This approach has the advantage that the free radicals are generated in-situ at the point of use. However, this approach presents a number of challenges.

In systems using liquid as a counter electrode, as well as in system using a solid counter electrode, it is challenging to maintain the generation of free radicals while avoiding sparks. Sparking is undesirable for a number of reasons, including that it may damage the electrodes. FIG. 2 is a graph showing a current-voltage curve for a gas discharge. V_{ignition} indicates the discharge voltage and V_{breakdown} indicates where electrical breakdown of the discharge gap occurs. Spontaneous transition of the non-thermal atmospheric pressure gas discharges (APGD) to the thermal discharge mode (spark) is one of the main issues of industrial applications of a gas discharge. The reason for such transition is the accumulation of too high charge in the discharge gap or on the surface of the electrodes, which becomes the seeds for the surge, uncontrolled growing in the number of charge particles, a local growing of the electrical field strength and the future acceleration of growing in the number of charge particles. In a stationary DC gas discharge, it is difficult to set a limit for such a transition, especially for a sharp multi-pin electrode geometry. The current I in FIG. 2 is the average current because the real current is a sequence of short current pulses corresponding to the propagation of individual electron avalanches (corona discharge) or streamers (APGD) in the discharge gap.

The main problem is that the amplitude of the breakdown voltage for every pin of multi-pin electrode system is different, and even for the individual pin, it fluctuates too much within a narrow range, and the amplitude of the breakdown voltage tends to drop over time. Because the current-voltage curve is close to flat (low slope) before the transition to the sparking mode, a small drop in the breakdown voltage leads to a big reduction in both the discharge current and discharge power, which leads to a significant drop in the number of ionized particles and radicals.

The common technique to prevent APGD from going into spark mode is the use of high velocity gas blowing. Due to the blowing off of the charge particles from the discharge gap, it makes it possible to control the transition of the APGD to the spark mode. Unfortunately, such a technic is not always acceptable, especially for the producing of the high radical concentration.

There are a number of techniques to attempt to prevent the transition from discharge to spark. One of them is the control of the voltage form applied to the discharge. If the applied voltage changes between V_{ignition} (Vₘᵢₙ) and V_{breakdown} (Vₘₐₓ) and the cycle time is sufficient to remove and/or neutralize the charge particles from the discharge gap, then every new discharge cycle will start under identical conditions for every electrodes of the multi-pin electrode system, which allows for stabilization of the amplitude of the breakdown voltage. FIG. 3 shows a schematic voltage profile and FIG. 4 is a graph showing measured voltage and current peaks for such an approach.

A liquid counter electrode system as in FIG. 1 has certain issues. One problem is ionic wind, i.e. the gas flow produced by the collision of charged particles with neutral gas molecules. This results in momentum transfer from the wind emerging from the sharp electrode to the flat water surface. As a result, a depression under the sharp electrode is formed on the surface of water. This is illustrated schematically in FIG. 5, where 10 is the discharge electrode, 16 is the undisturbed liquid surface, 22 indicates small amplitude waves in the surface, 24 indicates larger amplitude waves in the surface and 26 indicates the discharge. The depth of the cavity is proportional to the pressure of the ionic wind, which is proportional to the current from the discharge electrode 10. An increase in the cavity depth causes a drop in the electric field strength in the discharge gap, followed by a drop in the current and, as a result, the cavity depth decreases. This fluctuation in the depth of the cavity generates waves on the surface of the water. Waves from different pins interact with each other, forming unpredictable wave distribution over the water surface, which causes unpredictable changes in the length of the discharge gap. As a result conditions for sparking are created in some places. In addition, the ionic wind itself (having a speed in the range of 0.5-5 m/s) can also create waves on the water surface.

A second problem associated with a liquid counter electrode is the buildup of moisture due to electrostatic evaporation. The breakdown voltage usually decreases with an increase in humidity levels, and the breakdown voltage can easily fall below the applied voltage, especially considering the high humidity level in the discharge gap due to the electrostatic evaporation. FIG. 6 illustrates the combined impact of the evaporated water and the waves on the breakdown voltage and discharge current over time, as the gap between the liquid surface and the electrode decreases.

From the aforementioned it is apparent that there are problems with the generation of radicals with a high density and with high efficiency even when a liquid counter electrode is used. As such, new methods and devices are needed for effective generation of radicals.

### SUMMARY OF THE INVENTION

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

Certain embodiments of the present invention address some of the shortcomings of prior art free radical generators. In one example, a discharge electrode assembly is provided, having a plurality of discharge electrode pins in an array arranged in a radial pattern and electrically configured to receive one or more voltage pulses. A counter electrode may be a solid electrode or may be a liquid electrode. An exemplary liquid electrode includes a liquid bath configured to hold a liquid having an upper surface disposed at a liquid level separated from the discharge electrode pins by a discharge gap. In an example, a motive element is operable to rotate the discharge electrode assembly such that the array of discharge electrode pins rotates about an axis and the plurality of pins move relative to the counter electrode, such as the liquid in the liquid bath.

The rotational movement of the array of discharge electrode pins changes the discharge gap between each pin as the pin moves relative to the counter electrode, such as the liquid surface. This changing of the gap length is a technique for controlling electrical breakdown across the gap. This creates a cyclical variation of the discharge gap and may lengthen the streamers. This means that if the gap is short, the discharge condition approaches the breakdown voltage, and if the length of the discharge gap increases, then the electric field strength will decrease, and the rate of generation of charged particles will also become low, and the discharge will collapse/damp. A rotary electrode system may also have another advantage. Considering that the spark and arc discharge are tied to certain points on the electrodes, in case the discharge transitions into the spark/arc mode, the rotation will lead to the elongation of the spark/arc channel, reducing the voltage across the channel and, as result, the channel breakdown.

In some aspects, the liquid in the liquid bath has a controlled depth and the bath may have a porous bed. Further aspects are discussed below.

In an embodiment, a free radical generator includes a discharge electrode assembly with a plurality of discharge electrode pins in an array arranged in a radial pattern and electrically configured to receive one or more voltage pulses. A counter electrode has a surface separated from the discharge electrode pins by a discharge gap. A motive element is operable to rotate the discharge electrode assembly such that the array of discharge electrode pins rotates about an axis and the plurality of pins move relative to the counter electrode.

In an example, the counter electrode is a solid counter electrode. In a further example, the counter electrode is a liquid bath configured to hold a liquid having an upper surface disposed at a liquid level, the upper surface of the liquid defining the surface of the counter electrode.

In some versions, the plurality of discharge electrode pins each have one or more ignition tips, each ignition tip defined by an angle, each ignition tip positioned at a terminus of the respective discharge electrode pin proximal to the counter electrode.

In some versions, the array of discharge electrode pins includes a plurality of rows of the discharge electrode pins extending substantially perpendicular to a length of the discharge electrode assembly. The discharge electrode assembly may include a plurality of electrode elements, each element defining one of the rows of discharge electrode pins. Each subsequent row of discharge electrode pins proceeding in the length of the discharge electrode assembly may be angularly offset from a preceding one of the rows. Each of the electrode elements is an electrode disc.

In some versions, the discharge electrode assembly may further include a central rod and each row of the plurality of rows of the discharge electrode pins is a disc having a central hole and the discharge electrode pins extending therefrom. Each disc may be placed on the central rod to form the plurality of rows of the discharge electrode pins.

In some versions, the axis is a lengthwise axis and the plurality of discharge electrode pins extend generally perpendicularly to the lengthwise axis.

In some versions, the liquid bath is adjustable to adjust the liquid level, thereby adjusting the discharge gap.

In some versions, the discharge gap between a closest one of the discharge electrode pins

and the counter electrode surface is in the range of 0.5 to 30 mm.

In some versions, the liquid bath further has a porous bed. In further versions, the generator has a pump operable to pump the liquid into or out of the liquid bath to create a liquid flow relative to the discharge electrode assembly. The liquid flow may have a flow direction in the liquid bath, the flow direction being generally parallel to the axis of rotation of the discharge electrode assembly or perpendicular thereto.

In some versions, the generator has a circuit in electrical communication with the discharge electrode assembly and the counter electrode, the circuit operable to generate electrical pulses between the discharge electrode assembly and the counter electrode such that a plasma is created between tips of the discharge electrode pins and the counter electrode as the tips move relative to the counter electrode.

In some versions, the generator further has a reactor housing including a reactor tube, the discharge electrode assembly disposed in the reactor tube. The reactor tube may formed of a non-conductive material. The reactor housing may have gas inlets and gas outlets for a flow of gas through the discharge gap.

In an embodiment, a liquid treatment system includes a free radical generator according to any disclosed embodiment. The system may further have a gas flow generating element operable to flow a gas through the gap of the free radical generator and a gas introduction element operable to introduce the gas into a liquid to be treated. In some versions, the system further includes a liquid flow generating element operable to introduce a liquid into or remove a liquid from the liquid bath such that the liquid is treated.

In an example, the liquid is water.

In an embodiment, a method of cleaning a liquid includes providing any generator disclosed herein or any liquid treatment system disclosed herein, flowing a liquid to be cleaned through the liquid bath, and/or introducing a gas from the discharge gap into the liquid to be cleaned.

In an example, the method of liquid treatment includes a process of removing microplastics from liquid environments, further including agglomerating or precipitating the microplastic particles, and removing the agglomerated or precipitated particles, such as by filtration.

In an example, the method of liquid treatment is operable for disinfection against microbials and/or decontamination from chemicals and/or particulates.

In an embodiment, a gas treatment system includes a free radical generator as described herein, a gas flow generating element operable to flow a gas to be treated, a column in fluid communication with the gas flow generating element such that the flow of gas flows through an interior space of the column, and a liquid introduction element operable to introduce the liquid from the liquid bath into the interior space of the column such that the flow of gas is exposed to the liquid. The system may include a porous exchange bed disposed in the column. The liquid introduction element may include at least one sprayer for spraying the liquid in the interior space of the column. The system may include a reservoir for receiving the liquid introduced into the interior space after the flow of gas is exposed to the liquid and a recirculation element operable to move liquid from reservoir back to the liquid bath. A pump may be included for pumping the liquid from the liquid bath to the liquid introduction element. The system may further include a filter disposed such that the flow of gas passes through the filter after being exposed to the liquid, the filter being a hydrophilic or hydrophobic filter. The system may further include a chilled element disposed such that the flow of gas passes through the filter after being exposed to the liquid, thereby removing humidity from the flow of gas. The system may further include a chilling element operable to reduce a temperature of the liquid before the liquid is introduced into the interior space. The flow of gas may be a flow of air. The system may have an outlet for the flow of gas to exit the system after passing through the column. The outlet may be in fluid communication with the discharge gap such that a flow of free radicals is mixed into the flow of gas exiting the system.

In an embodiment, a system for treatment of an area is provided. The system includes a gas treatment system as described above wherein the outlet is in fluid communication with the discharge gap such that a flow of free radicals is mixed into the flow of gas exiting the system.

In an embodiment, a system is provided for treatment of an area. The system includes a free radical generator according to any embodiment herein and a gas flow generating element operable to flow a gas through the discharge gap, thereby introducing free radicals into the flow of gas.

In an embodiment, a dehumidifier, an air conditioner, or a ventilation system includes a gas treatment system according to any embodiment here.

In an embodiment, a method of fumigating a space or local surface treatment in the space, includes a system for treatment of an area or a gas treatment system as described above wherein the outlet is in fluid communication with the discharge gap such that a flow of free radicals is mixed into the flow of gas exiting the system.

In an embodiment, a method is provided for treatment of an area, including providing a system according to an embodiment herein and introducing the flow of gas with free radicals from the system into the space. The liquid in the liquid bath may contain one or more of: Iodine; Hydrogen Peroxide; Quaternary ammonium; L-Lactic Acid; Peracetic Acid; Citric Acid; Chlorine Dioxide; Isopropanol; Sodium Hypochlorite; or Ethanol.

In an embodiment, a method of treating a gas includes providing a generator or a system according to any embodiment herein and flowing a gas to be treated through the generator or system.

In an embodiment, a process of producing a radical in a fluid includes passing a fluid through the discharge gap of any embodiment of a free radical generator herein, applying a pulse voltage between the one or more discharge electrode pins and the counter electrode, the pulse voltage applied for a pulse time, and generating one or more streamers extending between the one or more discharge electrode pins and the counter electrode, the one or more streamers generating a free radical within the fluid. The fluid may be a gas and the gas may include oxygen and nitrogen. The radical may be a nitrogen radical or a hydroxyl radical.

In examples, the pulse voltage is 5 kV to 20 kV.

In examples, the process may further include applying a bias voltage between the discharge electrode pins and the counter electrode, the applying being successive voltage pulses. The bias voltage may be in the range of 1 to 500 V or 1 to 2000V.

In examples, the gas velocity is 0.1 m/s to 200 m/s, or optionally 5 m/s to 50 m/s.

In examples, the process may further include repeating the step of applying and the step of generating, the repeating defined by a pulse width, the pulse width from 10 nanoseconds to 50 microseconds, or optionally 400 nanoseconds to 1 microsecond.

In examples, the process may further include repeating the step of applying and the step of generating, the repeating defined by a pulse frequency, the pulse frequency from 100 Hz to 100 kHz, or optionally 10 kHz to 30 kHz.

In examples of the free radical generator, the discharge electrode pins each include 3 to 8 ignition tips.

In examples of the free radical generator, the discharge electrode pins each include a tip profile substantially perpendicular to a length of the discharge electrode pin, wherein the tip profile is in the shape of a triangle, a square, a pentagon, a hexagon, a heptagon, or an octagon.

In an embodiment, a system for fumigation of crops in an interior grow enclosure, includes an indoor grow enclosure configured to have crops disposed therein and an air treatment system according to any embodiment herein.

In an embodiment, a method for fumigation of crops in an interior grow enclosure includes providing an indoor grow enclosure with crops disposed therein, providing an air treatment system according to any embodiment herein, passing air through the system so as to introduce free radicals and/or other reactive species into the air passing through the system, and introducing the air with the free radicals and/or other reactive species into the indoor enclosure, thereby exposing the crops to the air with the free radicals and/or other reactive species.

In an embodiment, a system for sterilizing a device such as a medical instrument includes a housing having a receiving area for receiving the medical instrument, the receiving area including a liquid bath disposed therein such that the device received in the receiving area is at least partially positioned in the liquid bath. A discharge electrode assembly includes a plurality of discharge electrode pins in an array arranged in a radial pattern and electrically configured to receive one or more voltage pulses. A portion of the liquid bath defines a counter electrode having a surface separated from the discharge electrode pins by a discharge gap. A motive element is operable to rotate the discharge electrode assembly such that the array of discharge electrode pins rotates about an axis and the plurality of pins move relative to the counter electrode.

In an example, a dividing element separates the portion of the liquid bath defining the counter electrode from a portion of the liquid bath receiving the device for sterilization.

In an example, an impeller is configured for circulating the liquid bath.

In an embodiment, a method of sterilizing a device such as a medical instrument includes providing a system as described above, receiving the device in the receiving area, generating active species in the liquid bath with the discharge electrode assembly, thereby generating a sterilant liquid, and flowing the sterilant liquid over a surface of the device, thereby sterilizing the device.

### BRIEF DESCRIPTION OF THE DRAWINGS

FIG. 1 is a schematic illustration of a discharge electrode above a liquid counter electrode;

FIG. 2 is a graph of average current versus time;

FIG. 3 is a graph showing a voltage profile;

FIG. 4 is a graph showing measured voltage and current peaks;

FIG. 5 is a schematic illustration showing waves due to discharge;

FIG. 6 is a graphical representation of the combined impact of evaporated water and waves on breakdown voltage and discharge current over time;

FIG. 7 is a schematic representation of a rotating discharge assembly;

FIG. 8A is a schematic representation of a rotating discharge assembly;

FIG. 8B is graphical representation of more consistent discharge current;

FIG. 9 is an exploded view of a free radical generator according to an embodiment of the present invention;

FIG. 10 is an assembled cross sectional view of the generator of FIG. 9;

FIG. 11 is a detailed view of an end portion of a discharge electrode assembly surrounded by a reactor tube;

FIG. 12 provides a perspective view of six discharge electrodes organized on a central rod;

FIG. 13 is an illustration of pin offsets which may be used with some embodiments;

FIG. 14 provides three illustrations of porous beds that may be provided in a liquid bath;

FIG. 15 is a perspective view of a free radical generator with a moisture removal system according to an embodiment of the present invention;

FIG. 16 is a cross sectional view of the generator and system of FIG. 15;

FIG. 17 is a graph providing test data;

FIG. 18 is a graph providing additional test data;

FIG. 19 is a graph providing data on effectiveness;

FIG. 20A is a cross sectional view of an air treatment system according to an embodiment of the present invention;

FIG. 20B is a partially cutaway perspective view of an alternative embodiment of an air treatment system according to the present invention;

FIG. 20C is a chart with dehumidification data;

FIG. 21 is a view showing various element for use in a packed bed;

FIG. 22 is a graph providing test data;

FIG. 23 is graph providing additional test data;

FIG. 24 is a partially cutaway perspective view of a system for sterilizing a device, in accordance with an embodiment of the present invention;

FIG. 25 is a graph providing test data; and

FIG. 26 is a schematic representation of system for fumigation of indoor crops in accordance with an embodiment of the present invention.

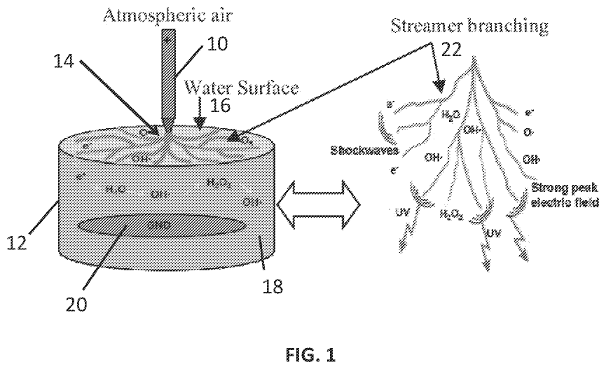

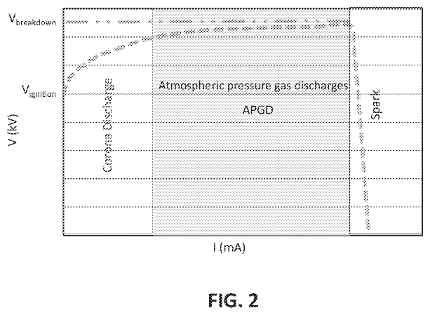

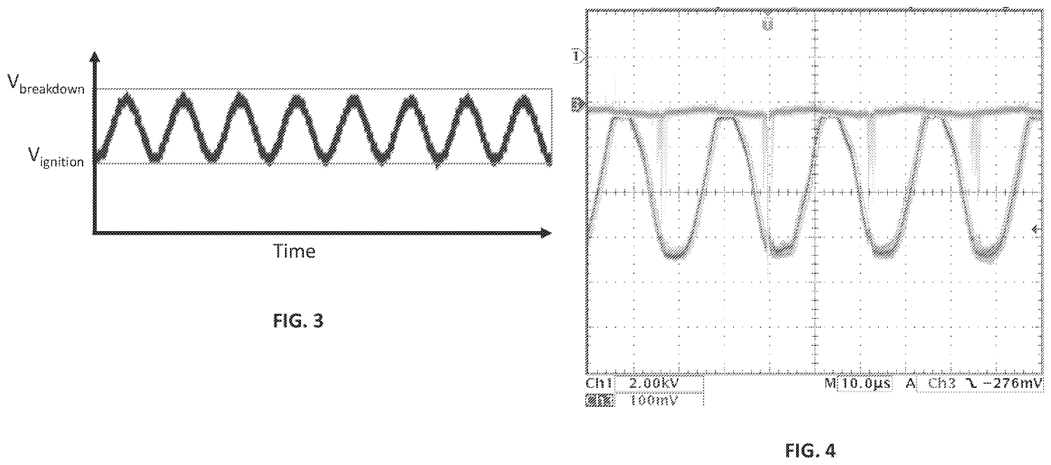

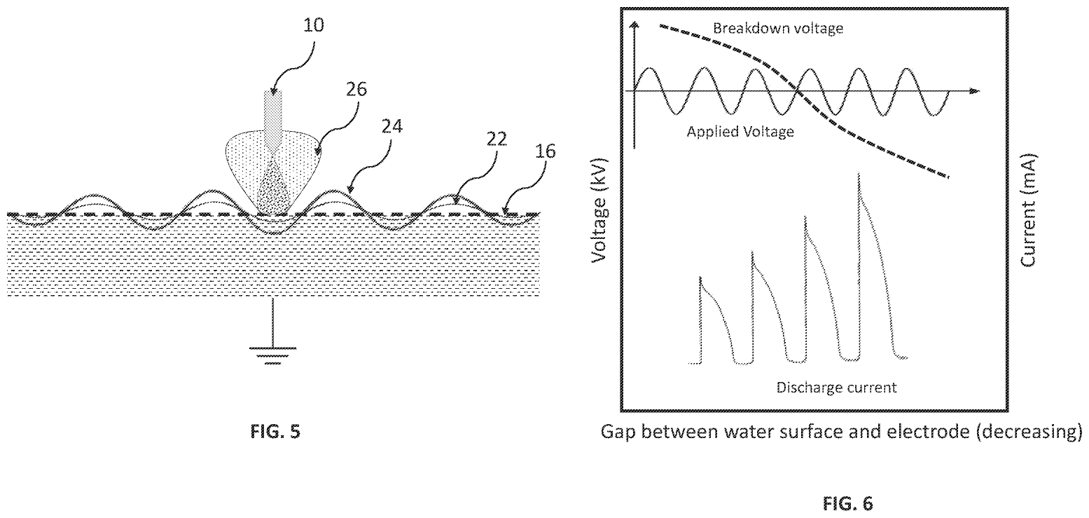

### DETAILED DESCRIPTION OF THE INVENTION

Detailed aspects are disclosed herein; however, it is to be understood that the disclosed aspects are merely exemplary in nature and may be embodied in various and alternative forms. The figures are not necessarily to scale. Therefore, specific details disclosed herein are not to be interpreted as limiting, but merely as a representative basis for any aspect of the invention and/or as a representative basis for teaching one skilled in the art to variously employ the present invention.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Throughout this specification, where publications are referenced the disclosures of these publications in their entireties are hereby incorporated by reference into this application to more fully describe the state of the art to which this invention pertains.

The following terms or phrases used herein have the exemplary meanings listed below in connection with at least one aspect:

A “dielectric” material as used herein is a medium or material that transmits electrical force without conduction and as such has low electrical conductivity. An illustrative example of a dielectric material is glass.

“Discharge space” as used herein means the gap between the active electrode and the ground electrode.

“FRG” as used herein means “Free Radical Generator” operating according to the teachings of this disclosure.

“Carbonaceous material” as used herein includes graphite, woven carbon or graphite fiber filled with binders, graphitized carbon materials, and compacted carbon materials, among others.

“Mist” as used herein includes a cloud of tiny droplets of a liquid suspended in a gas wherein droplet weight is lower than the drag force exerted by the gas.

“Fumigation” as used herein includes applying a gaseous fume of certain radicals to disinfect or to rid of biological organisms or toxins.

“Superbugs” as used herein includes a strain of bacteria, virus or fungi that has become resistant to one or more antibiotic drugs and other medications commonly used to treat infections it causes.

“Toxins” as used herein includes an antigenic poison or venom of plant or animal origin, optionally one produced by or derived from microorganisms and causing disease when present at low concentration in the body.

“Streamer” means a self-sustained ionization wave having substantial field enhancement in the range of 100-250 kV·cm⁻¹ and propagating in neutral gas which is converted into low-temperature plasma behind the wave front, resulting in a channel like appearance. The interior of the streamer channel consists of a conducting plasma with roughly the same electron and ion densities.

“Free radical” means an atom or group of atoms that has an unpaired valence electron and is therefore unstable and highly reactive as those terms are recognized in the art. For example, free oxygen radicals are produced by following inelastic electron collisions:

$${O}_{2} + {e}^{-} \to {O}^{+} + O + 2 {e}^{-} \qquad ( 1 )$$

$${O}_{2} + {e}^{-} \to O + O + {e}^{-} \qquad ( 2 )$$

$${O}_{2} + {e}^{-} \to {O}^{-} + O \qquad ( 3 )$$

which are expressed in a generic form as: O₂+e⁻→O*+O*. Other radicals may be produced by similar inelastic collisions depending upon the composition of the gas in the discharge space, such as:

$${H}_{2} O + {e}^{-} \to {OH}^{*} + {H}^{*} \qquad ( 4 )$$

$${N}_{2} + {e}^{-} \to {N}^{*} + {N}^{*} . \qquad ( 5 )$$

“Field” means the electric field, which can be positive or negative in nature. Similar fields repel each other and opposite fields attract each other.

“Motive element” means any structure operable to provide motion. Typical examples include an electric motor operable to provide rotation of another structure.

Certain embodiments of the present invention address some of the shortcomings of

prior art free radical generators. In one example, a discharge electrode assembly is provided, having a plurality of discharge electrode pins in an array arranged in a radial pattern and electrically configured to receive one or more voltage pulses. A counter electrode may be a solid electrode or may be a liquid electrode. An exemplary liquid electrode includes a liquid bath configured to hold a liquid having an upper surface disposed at a liquid level separated from the discharge electrode pins by a discharge gap. In an example, a motive element is operable to rotate the discharge electrode assembly such that the array of discharge electrode pins rotates about an axis and the plurality of pins move relative to the counter electrode, such as the liquid in the liquid bath.

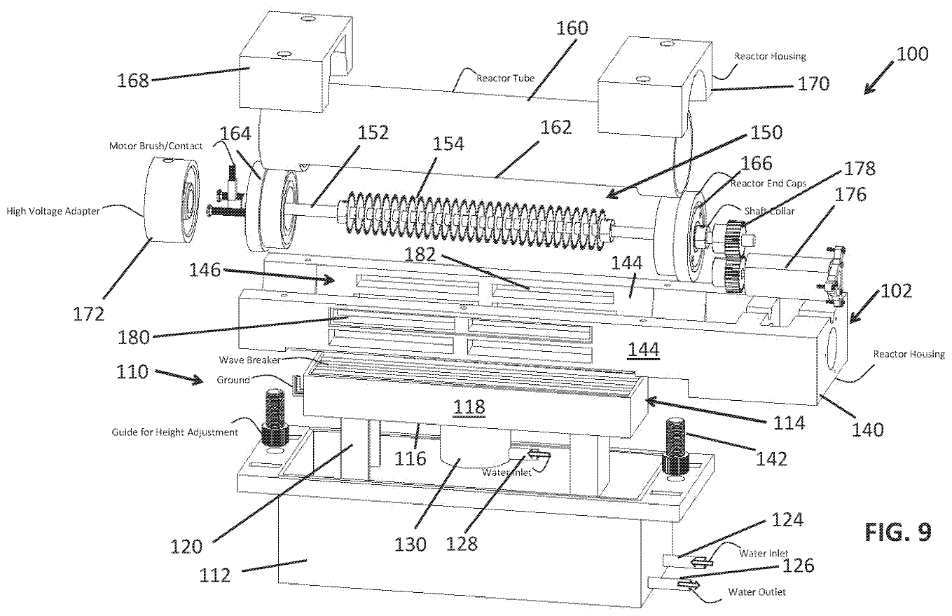

FIG. 7 schematically illustrates how a rotating discharge assembly interacts with a counter electrode; in this example the counter electrode is a solid counter electrode. A discharge electrode assembly 30 includes a plurality of discharge electrode pins 32 arranged in an array 34 arranged in a radial pattern. In this example, the array 34 of electrode pins 32 form a generally star-shaped element. The star-shaped element is supported for rotation about an axis A, with an arrow indicating clockwise rotation about the axis A. A counter electrode takes the form of a flat plate 40 with an upper surface 42 which is separated from the discharge electrode pins 32 by a discharge gap 46. In this illustration, the gap 46 is illustrated as the distance between the pin closest to the surface 42 and the surface. Because the array 34 is rotating, the discharge gap for each pin changes as the pin moves relative to the surface. This changing of the gap length may help electrical breakdown across the gap. This creates a cyclical variation of the discharge gap. This means that if the gap is short, the discharge condition approaches the breakdown voltage, and if the length of the discharge gap increases, then the electric field strength will decrease, and the Charged particle density will also become low, and the discharge will collapse/damp. The drawing at the left side of FIG. 7 shows one pin 32 at the position closest to the surface 42, when the gap 46 is smallest, with a discharge shown at 48. The drawing in the middle shows how the gap 46 lengthens as the array 34 rotates, and the discharge 48 is shown as being stretched. The drawing at the right side of FIG. 7 shows the array 34 further rotated and the gap 46 further lengthened. At this point, the next pin is closer to the surface 42 and the first discharge will collapse and a new discharge will form between this closer pin and the surface. In some examples, the discharge may extend from more than one pin at the same time. While FIG. 7 illustrates a flat and solid counter electrode, the rotating discharge electrode assembly will also provide benefits with other counter electrodes, including a liquid counter electrode.

A rotary electrode system may also have another advantage. Considering that the spark and arc discharge are tied to certain points on the electrodes, in case the discharge transitions into the spark/arc mode, the rotation will lead to the elongation of the discharge channel, reducing the voltage across the channel and, as result, the channel breakdown.

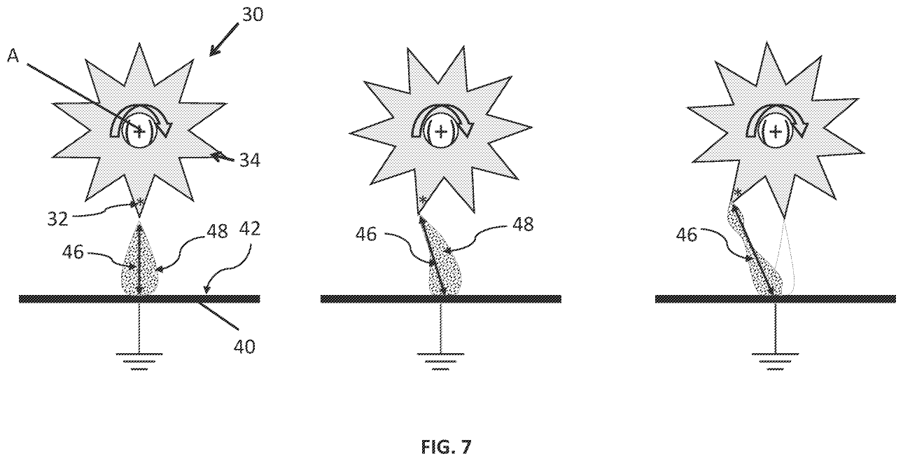

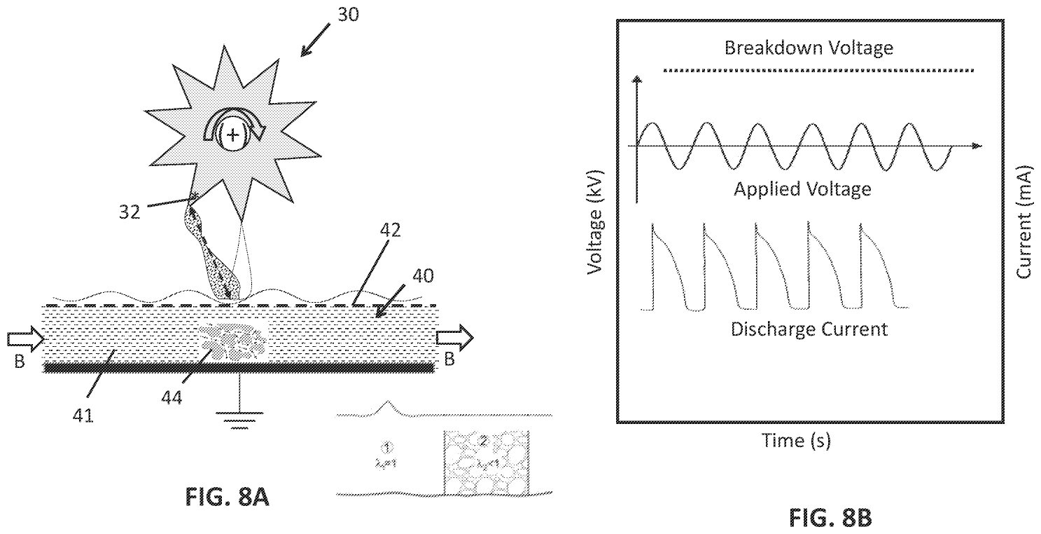

As discussed above with reference to FIGS. 5 and 6, a challenge with a liquid counter electrode is the formation of waves on the liquid surface, which changes the discharge gap and may lead to breakdown. The use of the rotary electrode system may also help to address this challenge. The rotational speed of the rotary electrode system will impact how the discharges interact with the surface and the rotational speed may be chosen so as to reduce or minimize the formation of waves.

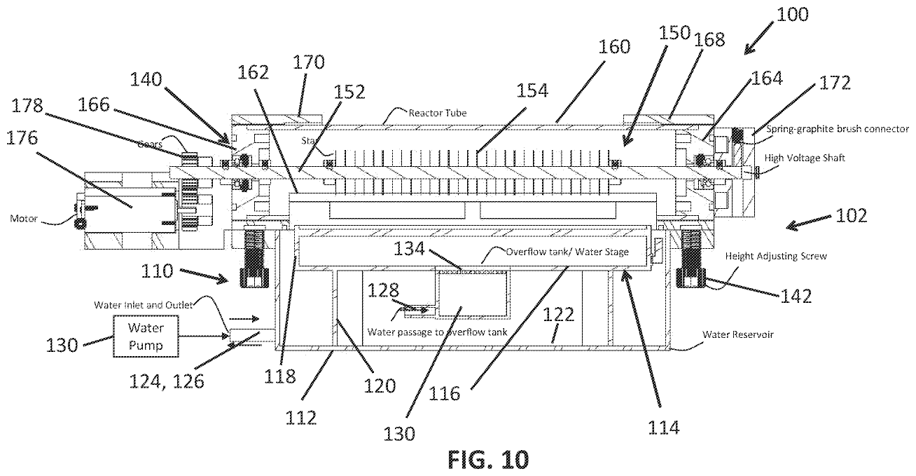

Further steps may be taken to further control wave formation. FIGS. 8A and 8B schematically illustrate these steps. The discharge electrode assembly 30 is positioned above the liquid bath 40 but the liquid bath includes a porous bed 44 disposed in the liquid 41 in the bath 40. The porous bed may take any of a variety of forms. Examples include ceramic or glass beads, a grid-like structure, a foam bed and/or a wavy ribbed structure in the liquid. Any porous bed providing the desired function, which is known to those of skill in the art or yet to be developed, may be used. Additionally, the depth of the liquid 41 is reduced or minimized. Optionally, the depth of liquid is between 0.01 mm and 1000 mm. Optionally, the depth of liquid is 1 mm, 5 mm, 10 mm, 20 mm, 30 mm, 40 mm, 50 mm, 60 mm, 70 mm, 80 mm, 90 mm and 100 mm. Optionally, the depth of liquid is between 1 mm and 10 mm. The combination of reduced depth (from the top of the porous bed to the liquid surface) and the presence of a porous bed reduce wave propagation. FIG. 8A illustrates the porous bed 44 being disposed in an area only directly below the electrode assembly 30. Alternatively, the porous bed 44 may occupy a larger area or the entirety of the liquid bath. Generally, the porous bed will be below the liquid surface 42 but alternatively the porous bed may partially extend above the surface.

Additionally, the liquid 41 may flow relative to the electrode assembly 30, as indicated by arrows B. In this example, the liquid flows in a direction opposed to the direction of movement of the pins 32 as they rotate clockwise. In a further example, the liquid flows in a direction generally parallel to the axis of rotation. Other flow directions are also possible.

The rotating electrode assembly 30 also acts as a fan to push evaporated water out of the discharge gap, further reducing the likelihood of breakdown.

The use of some or all of these steps may provide for improved performance. FIG. 8B illustrates a more consistent pattern of discharge current due to the fact that the breakdown voltage remains above the applied voltage.

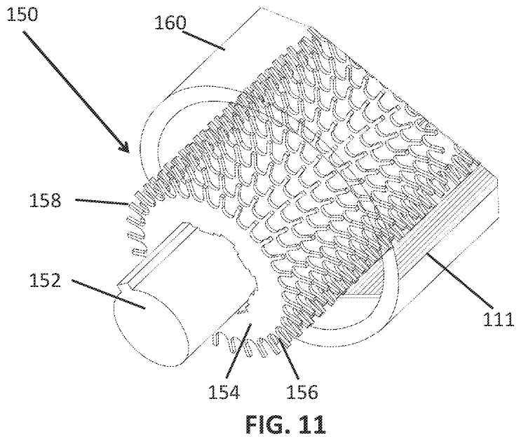

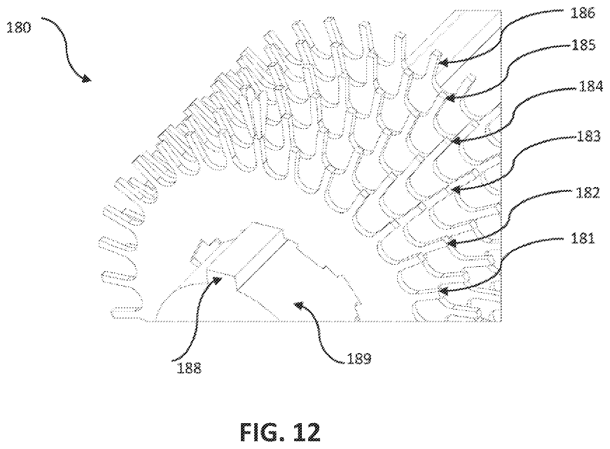

Referring now to FIGS. 9 and 10, an embodiment of a free radical generator 100 will be described. FIG. 9 is an exploded view of the generator and FIG. 10 is a cross-sectional view. The generator 100 includes a reactor housing 102 that generally defines the outer surfaces of the generator, though the generator may also be enclosed in a further device or housing. A liquid bath 110 may form a lower portion of the housing 102. In this example, the liquid bath 110 has a generally rectangular outer portion 112 and a liquid stage 114 supported in the outer portion 112. The liquid stage 114 holds a liquid to define the counter electrode for the generator 100. The liquid stage 114 takes the form of an elongated generally rectangular tray having a bottom 116 and upstanding sides 118. In one example, all of the upstanding sides have the same height, and the liquid level for the counter electrode is defined as a liquid level where the liquid overflows the sides 118. In other words, the height of the sides 118 defines the liquid level; if additional liquid is added, some of the liquid overflows the sides 118. The liquid stage is supported on legs 120 in the outer portion 112 such that the bottom 116 is spaced from a lower surface 122 of the outer portion 112.

In one example, liquid is continuously pumped into the liquid stage such that the liquid level is maintained at the height of the sides 118. A liquid inlet 124 and liquid outlet 126 are provided in the outer portion 112. The liquid stage likewise has a liquid inlet 128, which is fluidly connected to the liquid inlet 124 in the outer portion 112. A pump 130 continuously pumps liquid into the liquid stage 114, through the inlets 124 and 128. In the illustrated embodiment, the liquid inlet 128 of the liquid stage is connected to a receiving chamber 132 below a center region of the bottom 116 of the stage, with a grid or screen 134 between the chamber 132 and the remainder of the stage. Liquid flows into the chamber and then up into the stage 114 where it then flows out and causes continuous overflow of the stage 114 to maintain the liquid level. The overflowing liquid falls into the outer portion 112 and exits via the liquid outlet 126.

A main housing 140 is disposed on top of the liquid bath 110. The liquid bath may be connected to the main housing by height adjusting screws 142. As will be clear to those of skill in the art, by adjusting the screws 142, the position of the liquid bath, and therefore the position of the liquid surface defining the counter electrode, may be adjusted to change the discharge gap. Alternatively, the relative positions may be adjusted in other ways, such as adjusting the position of the liquid stage within the outer portion.

The main housing 140 is elongated with a pair of side walls 144 extending parallel to the sides of the liquid stage 114, with the side walls 144 spaced apart and extending upwardly to define a receiving space 146 therebetween, for receiving the discharge electrode assembly.

In this example, the discharge electrode assembly 150 an elongated central rod 152 with plurality of discs 154 arranged thereon, in a spaced apart mutually parallel arrangement. Each disc 154 has a plurality of discharge electrode pins extending radially outwardly therefrom. The central rod 152 defines an axis of rotation of the electrode assembly. The discs 154 extend along the central rod 152 for a length generally corresponding to the length of the liquid stage 114 such that each disc utilizes the liquid surface as a counter electrode. The liquid stage may have liquid provided near the center of the stage, as described above. As such, the flow of the liquid in the stage will be from the center outward, such that the flow is generally in the longitudinal direction of the elongated central rod and the elongated direction of the liquid stage, generally parallel to the axis of rotation of the discharge electrode assembly. The liquid flow is therefore perpendicular to a tangent to the pin movement. That is, as the pins rotate to a position closest to the liquid surface, they are moving in a direction generally perpendicular to the direction of liquid flow. Alternative approaches to liquid flow may also be used.

In this example, the discharge electrode assembly 150 is disposed in a reactor tube 160 formed of a non-conductive material. In this example, the reactor tube 160 has a generally circular cross-section but is open at the bottom 162 so that the pins of the electrode assembly 150 may discharge to the liquid counter electrode. In further examples, the reactor tube may have other cross-sectional shapes, such as square, rectangular, or polygonal. The discharge electrode assembly 150 is rotationally supported in the reactor tube 160 by a pair of reactor end caps 164 and 166, which are received in opposing ends of the reactor tube 160. The reactor end caps include bearings for rotational support of the central rod 152. The reactor tube 160, the end caps 164 and 166, and the discharge electrode assembly 150 are received between the side walls 144 of the main housing and held in place by retaining members 168 and 170. An electrical contact assembly 172 is provided at one end of the discharge electrode assembly 150, and provides a high voltage supply to the discharge electrode assembly.

Rotation of the discharge electrode assembly 150 may be accomplished in any way known to those of skill in the art. A motive element provides rotation. In the illustrated embodiment, the motive element is an electric motor 176 operable to rotate the central rod 150 through gears 178. Other approaches may also be used. In examples, the motive element or electric motor is electrically isolated from the discharge electrode assembly. This may be accomplished in any way known to those of skill in the art. In one example, the gears are non-conductive, such as made of polymer. A non-conductive belt may be used between the motor and central rod. As a further example, a motive element is coupled to the discharge electrode assembly by a magnetic coupling with an air gap. As yet another example, a mechanical coupling may be used wherein a dielectric material separates two sides of the coupling.

In the illustrated embodiment, the side walls 146 of the main housing have inlet openings 180 in one wall and outlet openings 182 in the other wall, to allow for air or gas to flow into and out of the area between the discharge electrode assembly 150 and the liquid bath. Air or gas may be blown through this area to remove moisture and discourage breakdown of the discharge gap.

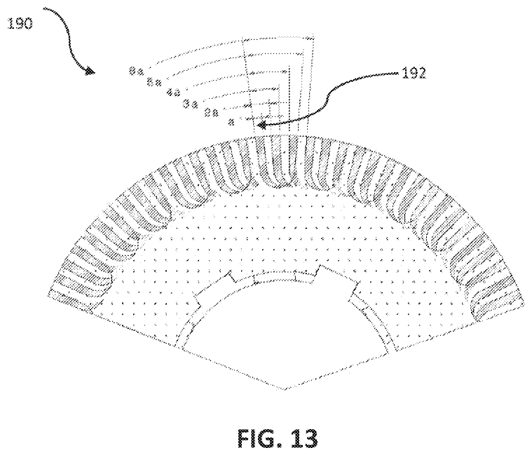

FIG. 11 provides a detailed view of an end portion of the discharge electrode assembly 150 surrounded by the reactor tube 160, with the reactor tube shown as transparent to make the structure easier to see. As discussed previously, the discharge electrode assembly 150 includes a central rod 152 and a plurality of discs 154. Each disc has a plurality of discharge electrode pins 156 extending radially outwardly, with each pin terminating in one or more ignition tips 158. The liquid surface is illustrated at 111

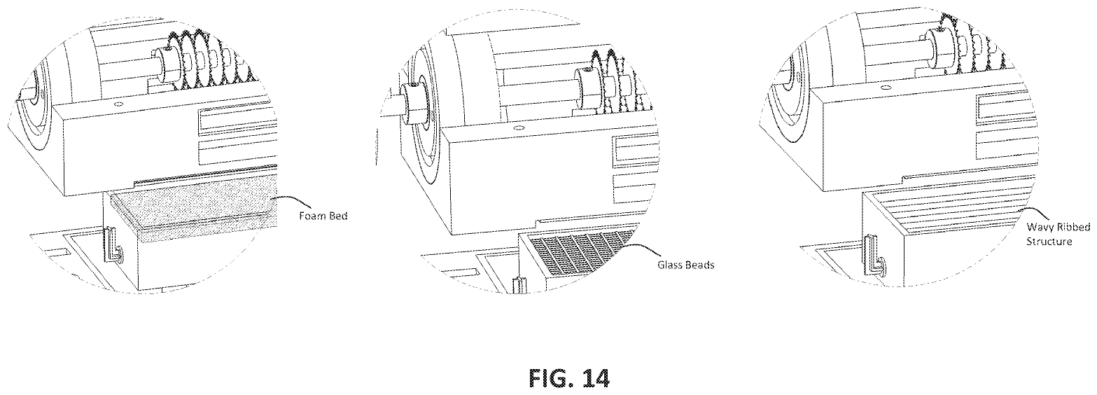

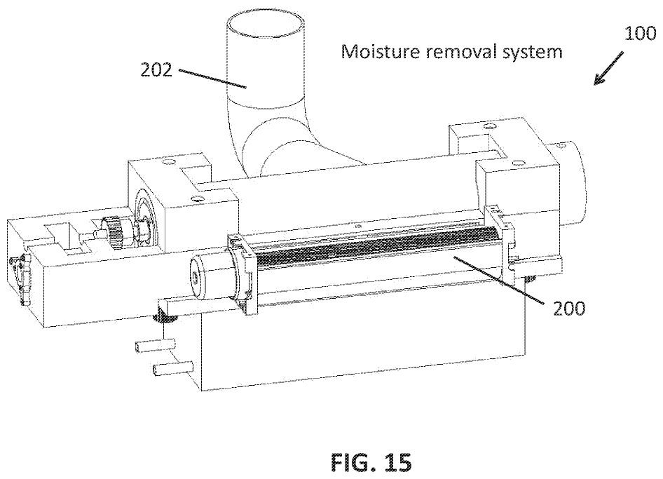

FIGS. 12 and 13 illustrate one approach to forming the electrode assembly. Each of the discs may be formed so as to engage a key on the central rod. FIG. 12 provides a perspective view of six discharge electrodes 181-186 organized on the central rod 189. The organization of the discharge electrodes are done according to a predefined pattern. Although one key is used to lock the discharge electrodes in place, the key slots in the discs enable the positioning of each electrode such that a desired angle between the discharge pins of the successive electrode with respect to the electrode in the previous row can be maintained. This is further illustrated in FIG. 13, where one cannot see through the inter pin gaps after six electrodes are assembled. In other words, if the pin width is “a”, the inter pin gap is “5a”, then the sixth electrode is a repeat of the first electrode. As such, a discharge pin with 0.25 mm×0.25 mm cross section would require an inter pin gap of 1.25 mm. The dimensional ratio of pin width to inter-pin gap may optionally vary and may not always stay at a 1:5 ratio. The important objective here is to position the discharge pins such that the distance between the streamers is kept uniform and they are uniformly distributed on the circumference of the discharge electrode assembly. This arrangement provides an obstruction to air flow through the inter-pin gap and maintains a suitable distance between the various pins. These electrodes can be conveniently precision cut by a laser beam or electron beam or stamped for mass manufacturing. In some aspects, the spacer of the discharge electrode is optionally between 0.5 and 20 mm, optionally between 1 mm and 10 mm, optionally between 1.5 mm and 5 mm. Accordingly, the inter pin gap is optionally between 0.5 and 20 mm, optionally between 1 mm and 10 mm, optionally between 1.5 mm and 5 mm. As such, the cross section of the pin is optionally between 0.05 mm×0.05 mm and 10 mm×10 mm, optionally between 0.1 mm×0.1 mm and 2.5 mm×2.5 mm. Alternatively, the pins may be arranged in other ways.

The electrode pins are illustrated as generally square in cross section with flat ends, such that the four corners define ignition tips. Other shapes and arrangements are also possible.

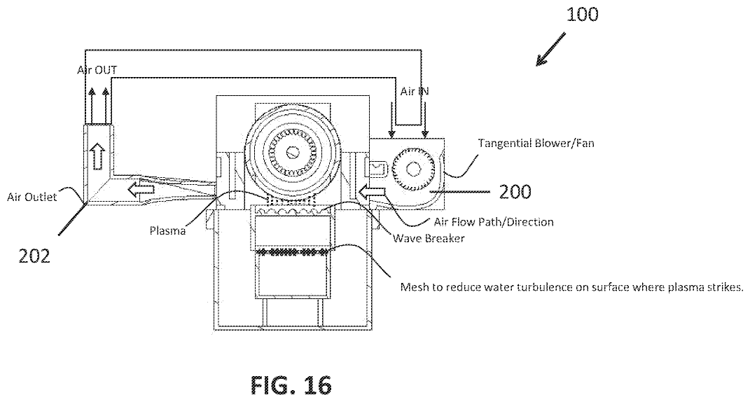

FIG. 14 illustrates three alternatives for providing a porous bed in the liquid bath. At the left, a foam bed is shown. In the middle, glass beads are shown. At the right, a wavy ribbed structure is shown. Other approaches may be used.

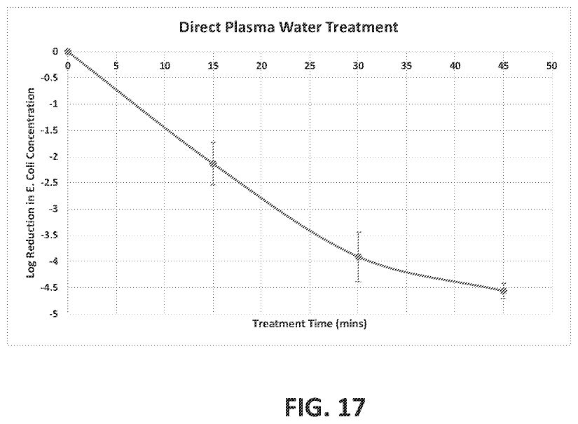

FIG. 15 illustrates a free radical generator 100 with a moisture removal system, including a blower 200 for blowing air into one side of the main housing, though the previously discussed inlet openings, and a duct 202 for removal of the moisturized air flowing out of the outlet openings of the main housing.

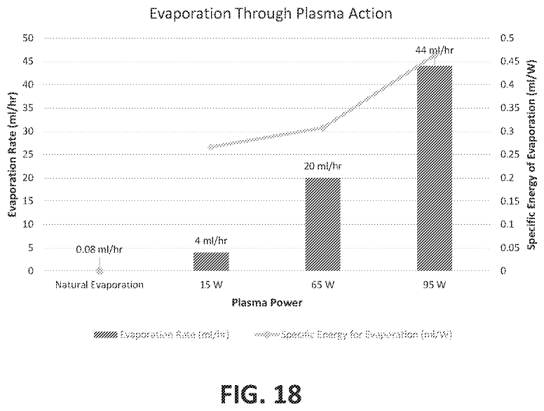

FIG. 16 provides a cross sectional view of the system including the blower 200, and further illustrates that the airflow may be recirculated. Alternatively, some or all of the water vapor may be released to the surrounding environment. As will be clear to those of skill in the art, the water vapor will include free radicals, and this vapor may be used for sanitizing. For example, water vapor including free radicals may be released into an unoccupied room for a period of time to sanitize surfaces in the room.

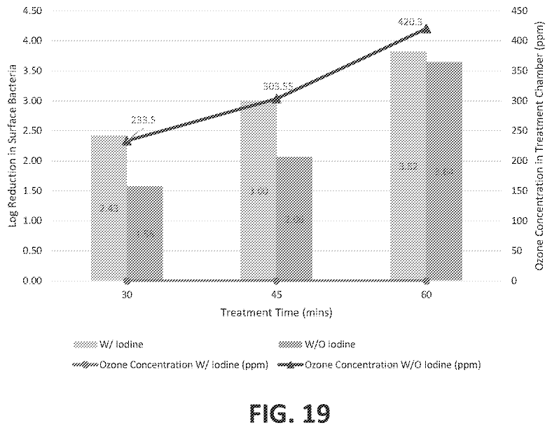

As will be clear to those of skill in the art, any of the embodiments of the present invention disclosed herein may also be used to clean polluted liquid, such as water including microbes. FIG. 17 provides test data for an exemplary system similar to the systems described above. In this example, 1.5 liters of de-ionized water was inoculated with 1 ml E. coli test cultures. The starting concentration of E. coli in the bulk water averaged between 7.05E5 to 1.88E6 CFU/ml. The 1.5 L of inoculated water was then circulated through the liquid stage for direct plasma treatment at a flow rate of 1 lpm. For the graph in FIG. 17, the water was then circulated through the system without energizing the electrode, and therefore without plasma discharge. The 0 minutes sample was then collected. Next, the electrode rotation and plasma power supply were started and samples were collected every 15 minutes thereon. The plasma power was ˜65 W, in positive polarity. The samples were serial diluted, plated and incubated for enumeration. The data presented in FIG. 17 us a composite of 3 tests and the error bars indicate the standard deviation. As shown, a greater than 4 log reduction was observed after 45 minutes of operation.

In some examples of a system for liquid treatment, the free radical generator includes a liquid bath as the counter electrode and the free radical generator creates free radicals in the liquid bath. The system for liquid treatment may include an additional pump for adding liquid to and/or removing liquid from the liquid bath such that the liquid is cleaned as it is passed through the liquid bath. As an alternative, the free radical generator generates free radicals in a gas and the gas is added to the liquid to be cleaned. This may utilize a solid counter electrode or a liquid counter electrode. As a further alternative, the above approaches may be combined, with liquid fed through the liquid bath and gas also introduced into the liquid. In examples, the liquid is water. A method of liquid treatment may include using such a system, adding gas to a liquid to be cleaned and/or passing liquid to be cleaned through the liquid bath.

The system as described above, and/or such as shown in FIGS. 9 and 10, has the ability to impact removal of microplastics from water sources, as part of or in addition to cleaning water of other contaminants. Owing to their size and long residence times, microplastics are considered a major ecological threat that traditional water treatment systems such as filtration cannot impact. The present invention directly impacts the suspension of microplastics by producing a change in the surface energy that accelerates the agglomeration and/or precipitation of the microplastic particles, enabling easy separation and collection through traditional filtration processes. A process of removing microplastics from liquid environments includes treating the liquid using a system as described above, agglomerating or precipitating the microplastic particles, and removing the agglomerated or precipitated particles, such as by filtration.

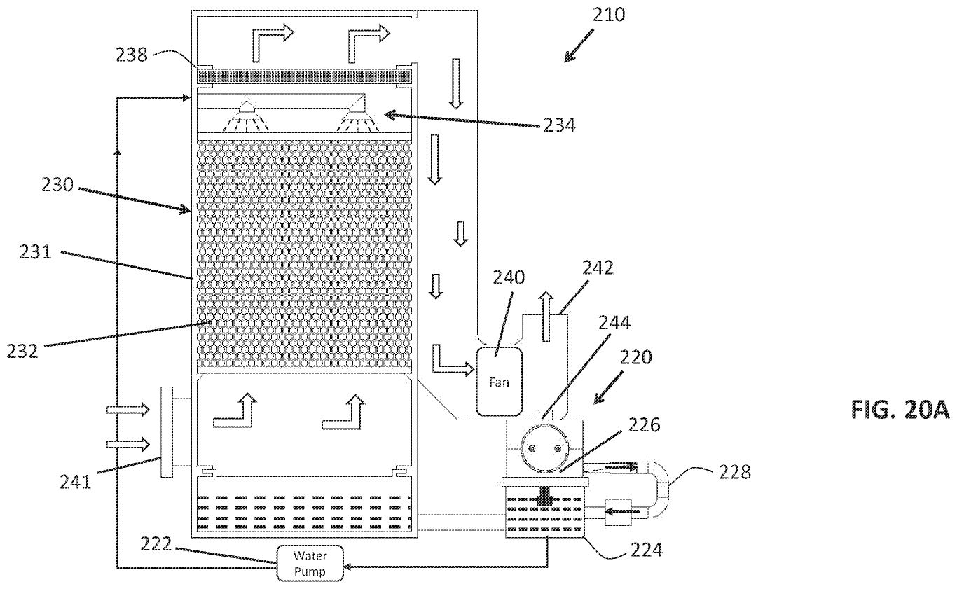

FIG. 18 provides test data for an exemplary embodiment showing liquid evaporation through plasma action. The tests were run by placing an entire direct plasma water treatment setup on a scale with a total measurement range of 5000 g and a resolution of 0.001 g. The system setup had an air flow rate through the direct plasma treatment zone of 12 m³/hr. The bar at the far left is with no plasma but with the electrode assembly rotating and the same air and water flow as in the tests with plasma. The remaining bars are for increasing plasma power levels. Weight loss over set periods of time were recorded and the evaporation rate in ml/hr was calculated. FIG. 18 illustrates that the use of plasma provides for an energy efficient evaporation of water, with the energy used being significantly less than boiling.

For certain embodiments of the present invention, the liquid of the liquid counter electrode is water, which may be deionized or otherwise treated. According to further embodiments of the present invention, various additives may be present in the liquid, with the liquid typically being water. Non-limiting examples of additives include one or more of the following:
1. Iodine
2. Hydrogen Peroxide
3. Quaternary ammonium
4. L-Lactic Acid
5. Peracetic Acid
6. Citric Acid
7. Chlorine Dioxide
8. Isopropanol
9. Sodium Hypochlorite
10. Ethanol

A combination of one or more of the above could also be possible; as a non-limiting example, the combination of Quaternary ammonium and Isopropanol. The use of one or more additives may be useful for surface treatment and for other purposes.

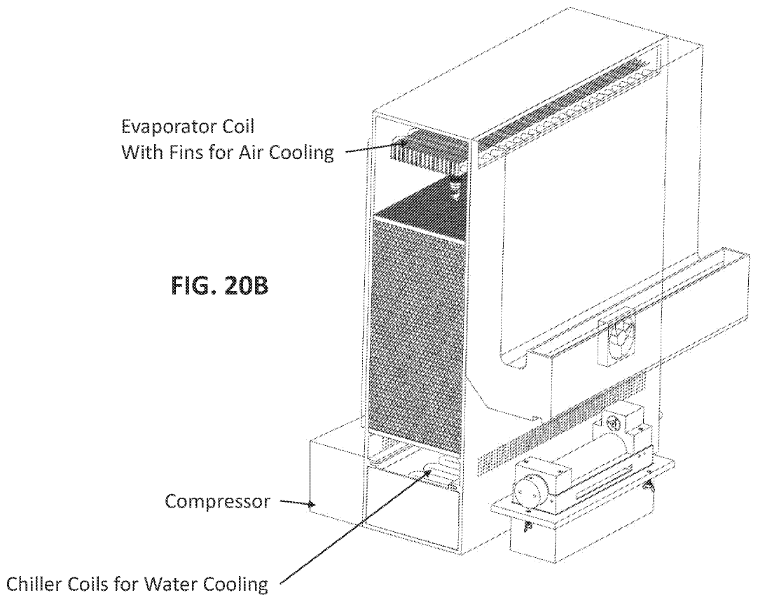

As discussed above, a system such as shown in FIG. 16 may be used for surface treatment with expended water vapor, containing free radicals. FIG. 19 provides data representing the effectiveness of an exemplary system. For this test, a direct plasma system was used with 1.5 L of de-ionized water inside a test chamber with a volume of approximately 700 liters. In one test case, 30 ml of Lugol's reagent (aqueous iodine) was added to the de-ionized water base. In another test case, plain de-ionized water was used. 1″×1″ aluminum coupons were inoculated with 20 ul of Pseudomonas aeruginosa and dried under air flow in sets of 2 in petri dishes. These coupons in petri dishes were aseptically transferred to the test chamber and placed at different locations at varying heights. The test was started by switching on the plasma power supply. The power of the system was approximately 65 W, running under a positive polarity condition. At pre-determined time points, some coupon petri dishes were extracted from the chamber. The total run time of the test was 60 minutes, at the end of which, all remaining coupons were removed from the chamber. All coupons were recovered by transferring to sterile letheen broth and vortexing for 60 seconds. Following this, serial dilutions were prepared and plated on TSA plates. All samples were enumerated at 16-24 hours of incubation. During both test cases, ozone concentration within the chamber was noted.

The presence of iodine reduces or eliminates ozone in the treatment vapor, making use of the system possible with people present in a treatment area. As shown in FIG. 19, the system was highly effective at reducing the surface bacteria, achieving a greater than 3 log reduction in 60 minutes, with or without iodine. The use of iodine increased the initial effectiveness and the measured ozone concentration in the chamber was approximately zero.

As an alternative, sodium chloride may be added to the water, or a combination of iodine and sodium chloride. As a further alternative, lactic acid or peracetic acid may be added.

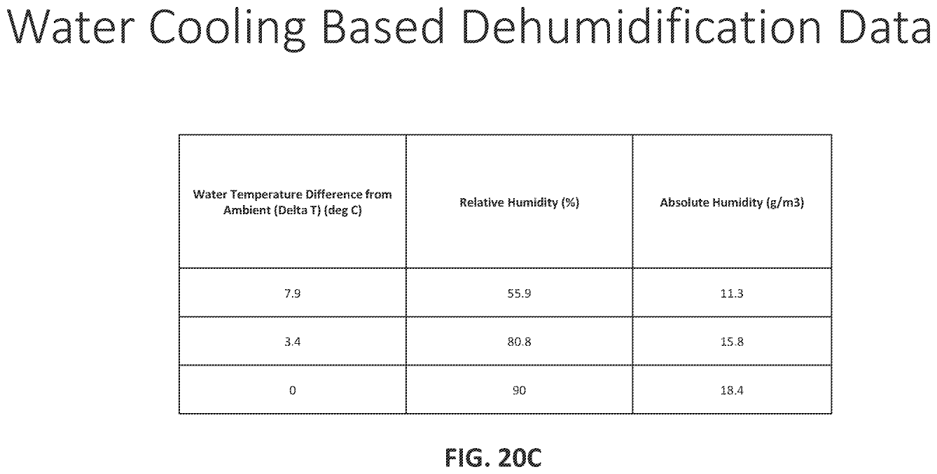

FIG. 20A illustrates an air treatment system 210 utilizing liquid that has been treated as discussed above. The air treatment system includes a free radical generator 220 similar to the earlier discussed embodiments. The air treatment system 210 further includes a scrubbing tower 230, which includes a generally vertical column 231 containing a porous exchange bed 232. The bed 232 is wetted with water from the free radical generator 220. In this example, a pump 222 pumps water out of the liquid bath 224 to sprayers 234 above the bed 232 such that the bed is wetted. The bed is porous such that gas may flow upwardly therethrough. An air inlet 236 is provided into the column 231 below the bed 232. The air then flows upwardly through the bed 232 where it interacts with the liquid. A filter 238 may be provided above the bed 232 with the air flowing through the filter. If low humidity is desired, the filter may be a hydrophobic filter and, if humidity release is desired, a hydrophilic filter may be used. The air then flows out into the surrounding area. In this example, the air is pulled through the column by a fan 240 and then exits via an air outlet 242. In this example, the air to be released to the surrounding area is not passed through the free radical generator, but instead enters through an air inlet 241 in the side of the column 231. Water from the porous bed 232 falls to the bottom of the column where it is collected and pumped back to the liquid stage for treatment. Dirt from the column may accumulate in the bottom reservoir and filtration may be provided for removal of larger particles. In one example, air is pumped from the discharge gap 226 of the free radical generator and into the liquid bath 224 by a fan and duct 228. In the above system, the air to be released to the surrounding area is not passed through the free radical generator. Alternatively, an opening or valve 244 may be provided between the free radical generator and the system, near the air outlet 242 for release of free radicals into the air, such as for the previously discussed surface treatment. As above, additives such as iodine, sodium chloride, lactic acid and/or peracetic acid may be added.

As an alternative to the above design, the scrubbing tower may be replaced with a spray tower, without the bed of porous exchange media. Instead, sprayers may be provided in a column to introduce liquid into the moving airstream, thereby exposing the flow of gas to the liquid.

Embodiments of the present invention may be used for air treatment. In an example, air moves through the scrubber portion of the device where a high exchange surface area is provided for water and air to interface. Contaminants (chemical, biological, particulate, etc.) are transferred from the air stream to the water. Treated air then exits the device, while the water is disinfected and decontaminated by the action of the free radical generator. Any of the embodiments described herein may be used for air treatment in this way.

FIG. 20B provides an alternative design to the system of FIG. 20A. In this design, a chilled element, such as an evaporator coil, is disposed such that the gas flowing through the tower passes through it after being exposed to the liquid. Excess humidity condensates on the chilled element and is removed from the flow of gas, thereby controlling the humidity level. The flow rate and duty cycle of the chilled element may be adjusted to achieve a desired humidity level.

This approach may be used with the packed bed or with the spray tower. FIG. 20B also has a chilling element, shown as chiller coils, for cooling the liquid that is sprayed into the tower. The chilling element may be disposed at any position in the liquid system to reduce the temperature. Reducing the temperature of the liquid reduces how much humidity is present in the flow of gas after it passes through the packed bed or spray tower. FIG. 20C provides exemplary data for the impact of water temperature difference on humidity levels for 30 degree Celsius air. At the bottom, the water is not chilled and the relative humidity is 90%. Above this, the water is chilled by 3.4 and 7.9 degrees Celsius and the resulting relative humidity is reduced to 80.8% and 55.9% respectively. Any combination of the chilling element, the chilled element and/or the filter may be used for controlling humidity. As a further aspect of the present invention, the free radical generator or the system of FIGS. 20A or 20B may form part of a dehumidifier, air conditioner or ventilation system. If such a system includes a chilled element, such as evaporator coils, the coils may provide for dehumidification.

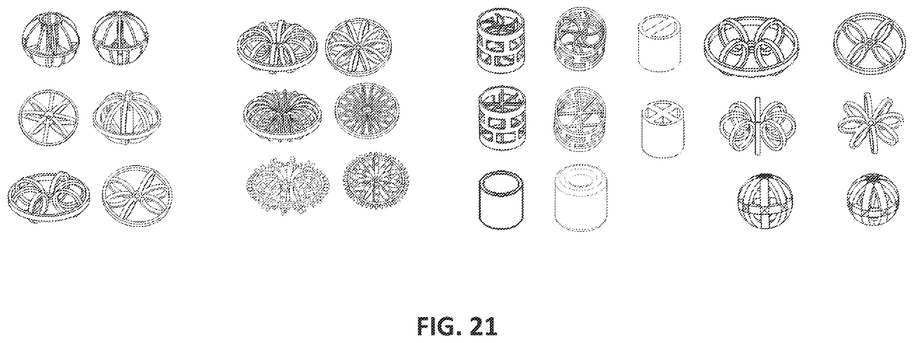

As known to those of skill in the art, the porous exchange bed may be constructed in a variety of ways, as long as it allows interaction between a wetted surface and the airflow. FIG. 21 illustrates various elements for use in a packed bed. The bed may utilize several types of packing media including Raschig Rings, Pall Rings, Tri-packs, Tellerette rings, etc. The media type may be selected to achieve the best balance between total volume of media used, total surface area afforded and resistance to air and water flow. In other aspects, a combination of media types in various proportions may be used to achieve the stated balance above. It is highly desirable to select media materials with good chemical compatibility with reactive oxygen and nitrogen species that are generated in the water through the action of the free radical generator. Materials may be selected from any of Polyethylene (PE), Polypropylene (PP), Reinforced polypropylene (RPP), Polyvinyl chloride (PVC), Chloride polyvinyl chloride (CPVC), Polyvinylidene fluoride (PVDF), Metal (SS), Ceramic or Graphite. More preferably, media materials may be any one or a combination of Polyethylene (PE), Polyvinyl chloride (PVC), Chloride polyvinyl chloride (CPVC), Polyvinylidene fluoride (PVDF), Ceramic or Graphite. The specific surface area of the media, defined as the nominal surface area available for a given volume of media, in the units of m²/m³ is an important parameter in selecting the media type and size and directly impacts the design efficiency of the scrubber. Specific surface areas ranging from 60 m²/m³ to 500 m²/m³ are desirable, with specific surface area above 200 m²/m³ being preferred. The packing density of the media defines the number of pieces of media that can be packed per a given volume. By extension, the packing density defines the total surface area available and influences the choice of media type selection. The higher the packing density, the more surface area available from the media, with a trade-off of more resistance to air and water flow. Packing density ranges from 500 l/m³ to 500,000 l/m³ may be employed. Packing density ranges from 50,000 l/m³ to 300,000 l/m³ are preferred. In another aspect, a combination of media with different packing densities may be employed to achieve a balance between total surface area afforded and restriction to air flow.

The ratio of water flow rate to air flow rate through the scrubber, defined as L/Q in units of liters/m³, is an important efficiency parameter. In general, a higher L/Q is preferred to achieve uniform wetting of the media and consequently a larger interface surface area for the water and the air. Values for L/Q may range from 0.5 liters/m³ to 20 liters/m³, preferably between 2 liters/m³ and 10 liters/m³. L/Q values beyond 20 liters/m³ require higher pumping requirements and water retention capacities whilst not offering a significant efficiency boost.

The residence time of air through the packed media bed impacts the capture efficiency of the scrubber, with longer residence time leading to higher efficiency. Residence time can range from 0.05 s to 5 s, with a preferred residence time between 0.1 s and 1 s. A variable in relation to the residence time that also impacts collection efficiency is the linear velocity of air through the packed media bed. The linear velocity range may be between 0.1 m/s and 1 m/s and preferably between 0.2 m/s and 0.6 m/s.

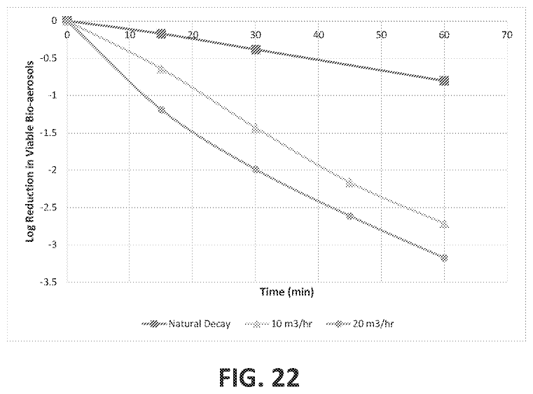

FIG. 22 provides test data for a wet packed-bed tower scrubber, as in FIG. 20A, with 2 liters of active media. The active media was either in the form of 12 mm×12 mm Raschig rings with a bulk surface area of 330 m²/m³ or 12 mm or Rosette rings with bulk surface area of 269 m²/m³. Water was sprayed onto the packed bed at a flow rate of 3 liters per minute (lpm) through a spray nozzle. The air flow rate was set to 10 m³/hr for the first set of tests and 20 m³/hr for the second set of tests, flowing counter to the direction of water flow. The scrubber system was placed inside a closed chamber with a volume of approximately 2 m³. E. coli cultures were nebulized into the test chamber for a duration of 5 minutes using a medical nebulizer, accumulating to a starting concentration of 7.5E5 to 3.6E6 CFU/m³. The nebulization was stopped and the air scrubber was switched on for a test duration of 60 minutes. At pre-determined time points, bio-aerosol samples were collected by drawing chamber air at a flow rate of 12.5 lpm into AGI-30 impingers containing 20 ml of sterile PBS water. Sampling duration was kept at 5 minutes. Following sample collection, serial dilutions were performed and plated in duplicate. Tests were similarly performed, without an air scrubber in the chamber to establish natural decay. All counts were performed at 24 hrs of incubation. As shown, the system significantly reduces the presence of viable bio-aerosols.

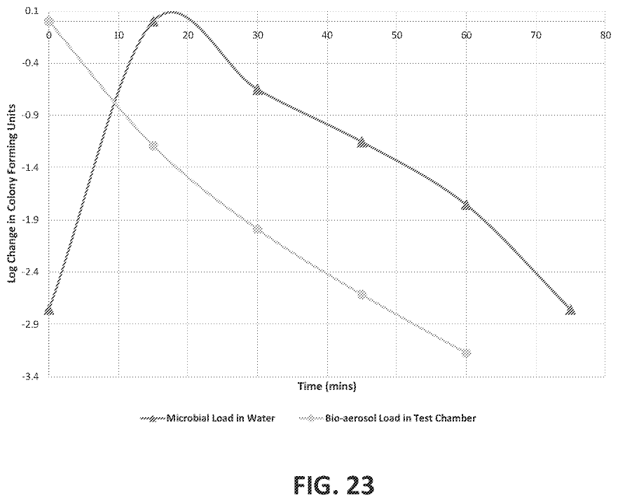

FIG. 23 provides data for a combination of air scrubbing and water treatment. Again, a wet packed-bed tower scrubber, as in FIG. 20A, with 2 liters of active media, was used. The scrubber's water loop was attached to a direct plasma treatment system. Plasma treated water was sprayed onto the packed bed at a flow rate of 3 lpm through a spray nozzle. Internal to the direct plasma system, another pump circulated the water through the plasma stage at a rate of 1.5 lpm. The air flow rate was set to 20 m³/hr through the scrubber. Prior to bacteria aerosolization, 5 liters of fresh de-ionized water was added to the system and circulated for 2 hrs at 3 lpm with plasma on to disinfect any bacteria present in the lines and test system. A water sample was collected after this and named water sample at time, T=0 mins. E. coli cultures were nebulized into the test chamber for a duration of 5 mins using a medical nebulizer, reaching a starting concentration of 7.5E5 CFU/m³. The nebulization was stopped and the air scrubber was switched on for a test duration of 60 mins. At pre-determined time points, bio-aerosol samples were collected by drawing chamber air at a flow rate of 12.5 lpm into AGI-30 impingers containing 20 ml of sterile PBS water. Sampling duration was kept at 5 mins. At the same time points and extending beyond the 60 mins, water samples were collected by sampling directly out of the water trough using a mechanical pipette. Following sample collection, serial dilutions were performed and plated in duplicate. All plates were counted 24 hrs after incubation at 37 deg C. Again, a significant reduction in bacteria was observed

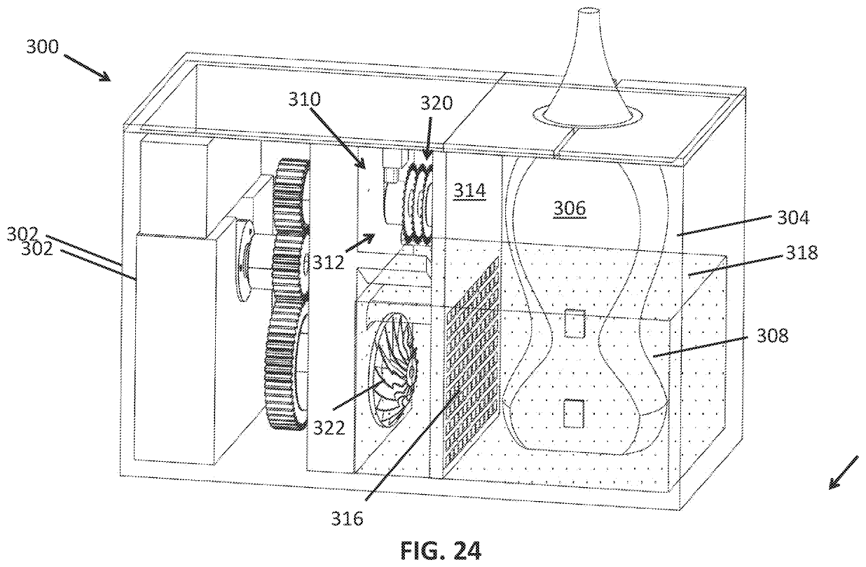

Referring now to FIG. 24, a further embodiment of the present invention will be described. A system 300 for sterilizing a device, such as a medical instrument, has a housing 302 with a receiving area 304 for receiving the device 306 to be sterilized. A sterilizing bath 308 is defined in the receiving area and the device 306 is positioned at least partially into this bath. A free radical generator 310 is provided in a generator area 312, which is separated from the receiving area 304 by a dividing wall 314. A liquid opening 316 is provided in the wall 314 below the surface 318 of the liquid, such that liquid may pass between the receiving area and the generator area, but such that air does not generally pass. The generator 310 includes a discharge electrode assembly 320 disposed in the generator area 312 above the liquid level. It operates as previously discussed, using the liquid as a counter electrode. An impeller 322 is provided in the liquid in the generator area and causes circulation of the water. Dividers 324 may be provided to cause flow of liquid relative to the discharge electrode of the free radical generator. The liquid in the sterilizing bath may have one or more of the above-discussed additives and/or a detergent. The free radical generator may include any of the features or elements described herein for use with the other embodiments.

In an example, the sterilizing bath is filled with a liquid, commonly water. The free radical generator processes the liquid, creating active species in situ with sterilant characteristics. The instrument requiring sterilization is then placed in the sterilization bath. The mechanical flow of the sterilant liquid across the instrument surfaces for a prescribed time period achieves sterilization of the instrument.

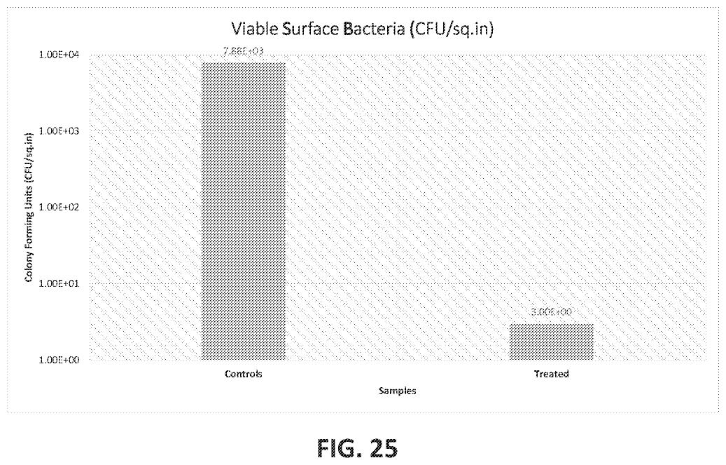

FIG. 25 provides data for an exemplary system similar to in FIG. 24. 200 ml of fresh deionized water was added to to the direct plasma test reservoir. The device was run for 120 mins until the water was ‘charged’. Prior to the test, several 1″×1″ aluminum coupons were inoculated with 20 ul of Pseudomonas aeruginosa and dried in an incubator at 37 C. After the device was run for 120 mins, 20 ml of the treated water was transferred to a 50 ml conical vial. Immediately, one inoculated aluminum coupon was dropped into the vial and vortexed at 2000 rpm for 60 s. The transfer and vortex process was repeated for several coupons. For controls, inoculated coupons were similarly vortexed in deionized water. Dilutions of the vortexed water were prepared and 100 ul of each sample was plated on TSA plates. All samples were incubated at 37 C for 16 hours before counting. A net log reduction of 3.42 log was noted in the plasma water treated samples.

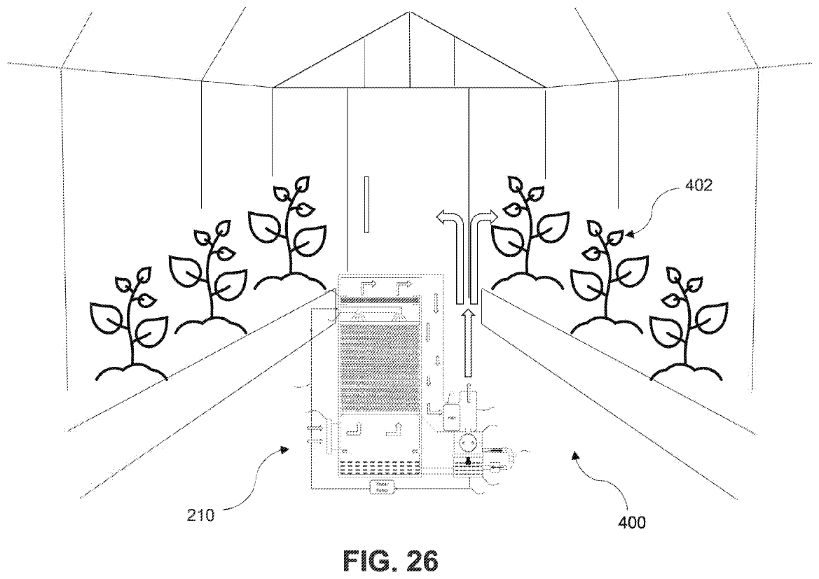

FIG. 26 illustrates an embodiment of the present invention for fumigation of crops in indoor growing environments including but not limited to greenhouses, cannabis grow houses, vertical farms and hydroponics. An indoor grow enclosure is generally shown at 400 with plants 402 represented schematically. Fungal species, particularly mold and powdery mildew impact several species of plants and are a major threat to crop yield in all indoor farming. Utilizing an air treatment system 210 such as that presented in FIG. 20A, the free radicals and other reactive species generated may be released into the indoor farming environments to disinfect the air and plant surfaces. Any other embodiment of the present invention may likewise be used in such a configuration. In addition, the free radical generator utilizing a liquid counter electrode system has the impact of transforming molecular nitrogen in the air into nitrate ions at the liquid interface, which can then be transported to the plants by the air movement through the device. Nitrogen in the form of nitrates (NO3-) is considered fixed nitrogen, an inorganic form of nitrogen that is readily usable by plants for biosynthesis. It will be apparent to those skilled in the art that such an application of embodiments of the present invention would facilitate faster plant growth and higher crop yields.

Those of skill in the art will recognize that the embodiments disclosed herein may be operated using a range of parameters, such as the speed of rotation of the discharge electrode assembly, the pulse frequency and characteristics, the speed and volume of gas flow, the gap size, etc. As non-limiting examples, the speed of rotation of the discharge electrode assembly may be in the range of 100 to 10,000 RPM, such as 200 to 5,000, or such as approximately 1000 RPM. The energy per pulse and the pulse frequency are two important parameters that determine the overall power consumption by the device. The energy dissipated per discharge pin may optionally be between 0.1 μJ to 100 μJ, optionally between 1.0 and 20.0 μJ. The frequency may optionally range between 100 Hz to 100 kHz, optionally between 10 kHz and 30 kHz.

The gas flow rate considerably influences the energy that can be delivered to the device and in turn the radical concentration as well as quantity. While low gas flow increases the radical concentrations, the yield is lowered due to competition between generation and destruction rates as well as space charge build up which leads to unstable ignition voltage and arcing. Higher gas flow reduces the radical concentration but improves the yield. For a given pressure gradient, the gas flow rate depends on the discharge gap, and hence, the gas velocity in the discharge space is a useful parameter for proper device operation. The gas velocity in the discharge space may optionally be in the range of 0.1 m/s to 200 m/s. As such, the gas velocity in the discharge space is optionally 2 m/s, optionally 5 m/s, optionally 10 m/s, optionally 15 m/s, optionally 20 m/s, optionally 30 m/s, optionally 40 m/s, optionally 50 m/s, optionally 60 m/s. The volumetric energy, i.e., energy/liter of gas, is an important characteristic of the device. The volumetric energy may optionally vary from 5 J/L to 5 KJ/L, optionally 200 J/L to 1 KJ/L.

The free radical generator may be operated with a bias voltage. The bias voltage is optionally between 0 and 500V compared to the actual pulse voltage (1 to 100 kV, optionally 5 to 20 kV, optionally 7 kV to 20 kV) and its magnitude depends on several parameters such as the gas flow rate and its composition, electrode design, and the applied voltage. Streamer discharge is also known to produce a gas flow. The ions within the discharge space are accelerated and, through collisions, the momentum of ions is transferred to neutrals, resulting in a gas flow. The effect of the bias voltage becomes less important when the gas velocity in the discharge space is high (>5 m/s), as the conductivity is reduced due to migration of space charge from the discharge space, more particularly from the ignition tips.

While aspects of the invention have been illustrated and described, it is not intended that these aspects illustrate and describe all possible forms of the invention. Rather, the words used in the specification are words of description rather than limitation, and it is understood that various changes may be made without departing from the spirit and scope of the invention.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified.

The foregoing description is illustrative of particular embodiments of the invention, but is not meant to be a limitation upon the practice thereof. It is the following claims, including all equivalents, which define the scope of the invention.

We claim:

1. A free radical generator, comprising:
a discharge electrode assembly comprising a plurality of discharge electrode pins in an array arranged in a radial pattern and electrically configured to receive one or more voltage pulses;
a counter electrode having a surface separated from the discharge electrode pins by a discharge gap; and
a motive element operable to rotate the discharge electrode assembly such that the array of discharge electrode pins rotates about an axis and the plurality of pins move relative to the counter electrode.

2. The free radical generator of claim 1, wherein the counter electrode is a solid counter electrode.

3. The free radical generator of claim 1, wherein the counter electrode comprises a liquid bath configured to hold a liquid having an upper surface disposed at a liquid level, the upper surface of the liquid defining the surface of the counter electrode.

4. The free radical generator of any one of claims 1-3, wherein the plurality of discharge electrode pins each comprise one or more ignition tips, each ignition tip defined by an angle, each ignition tip positioned at a terminus of the respective discharge electrode pin proximal to the counter electrode.

5. The free radical generator of any one of claims 1-4, wherein the array of discharge electrode pins comprises a plurality of rows of the discharge electrode pins extending substantially perpendicular to a length of the discharge electrode assembly.

6. The free radical generator of claim 5, wherein the discharge electrode assembly comprises a plurality of electrode elements, each element comprising one of the rows of discharge electrode pins.

7. The free radical generator of claim 5 or 6, wherein each subsequent row of discharge electrode pins proceeding in the length of the discharge electrode assembly is angularly offset from a preceding one of the rows.

8. The free radical generator of claim 6 or 7, wherein each of the electrode elements in an electrode disc.

9. The free radical generator of any one of claims 5-8, wherein the discharge electrode assembly further comprises:
a central rod; and
each row of the plurality of rows of the discharge electrode pins comprises a disc having a central hole and the discharge electrode pins extending therefrom;
wherein each disc is placed on the central rod to form the plurality of rows of the discharge electrode pins.

10. The free radical generator of any one of claims 1-9, wherein the axis is a lengthwise axis and the plurality of discharge electrode pins extend generally perpendicularly to the lengthwise axis.

11. The free radical generator of any one of claims 3-10, wherein the liquid bath is adjustable to adjust the liquid level, thereby adjusting the discharge gap.

12. The free radical generator of any one of claims 1-11, wherein the discharge gap between a closest one of the discharge electrode pins and the counter electrode surface is in the range of 0.5 to 30 mm.

13. The free radical generator of any one of claims 3-12, wherein the liquid bath further comprises a porous bed.

14. The free radical generator of any one of claims 3-13, further comprising a pump operable to pump the liquid into or out of the liquid bath to create a liquid flow relative to the discharge electrode assembly.

15. The free radial generator of claim 14, wherein the liquid flow has a flow direction in the liquid bath, the flow direction being generally parallel to the axis of rotation of the discharge electrode assembly or generally perpendicular to the axis of rotation.

16. The free radical generator of any one of claims 1-15, further comprising a circuit in electrical communication with the discharge electrode assembly and the counter electrode, the circuit operable to generate electrical pulses between the discharge electrode assembly and the counter electrode such that a plasma is created between tips of the discharge electrode pins and the counter electrode as the tips move relative to the counter electrode.

17. The free radical generator of any one of claims 1-16, further comprising a reactor housing including a reactor tube, the discharge electrode assembly disposed in the reactor tube.

18. The free radical generator of claim 17, wherein the reactor tube is formed of a non-conductive material.

19. The free radical generator of claim 17 or 18, wherein the reactor housing has gas inlets and gas outlets for a flow of gas through the discharge gap.

20. A liquid treatment system, comprising:
a free radical generator according to any of claims 1-19.

21. The liquid treatment system according to claim 20, further comprising a gas flow generating element operable to flow a gas through the gap of the free radical generator and a gas introduction element operable to introduce the gas into a liquid to be cleaned.

22. The liquid treatment system according to claim 21, further comprising liquid flow generating element operable to introduce a liquid into or remove a liquid from the liquid bath such that the liquid is treated.

23. The liquid treatment system according to claim 21, wherein the liquid is water.

24. A method of cleaning a liquid, comprising:
providing the generator according to any of claims 3-19 or the system according to any of claims 20-23; and
flowing a liquid to be cleaned through the liquid bath; and/or
introducing a gas from the discharge gap into the liquid to be cleaned.

25. The method of claim 24, wherein the method comprises a process of removing microplastics from liquid environments, the method further comprising agglomerating or precipitating the microplastic particles, and removing the agglomerated or precipitated particles, such as by filtration.

26. The method of claim 24, wherein the method is operable for disinfection against microbials and/or decontamination from chemicals and/or particulates.

27. A gas treatment system, comprising:
a free radical generator according to any of claims 3-19;
a gas flow generating element operable to flow a gas to be treated;
a column in fluid communication with the gas flow generating element such that the flow of gas flows through an interior space of the column; and
a liquid introduction element operable to introduce the liquid from the liquid bath into the interior space of the column such that the flow of gas is exposed to the liquid.

28. The gas treatment system of claim 27, further comprising a porous exchange bed disposed in the column.

29. The gas treatment system of claim 27 or 28, wherein the liquid introduction element comprises at least one sprayer for spraying the liquid in the interior space of the column.

30. The gas treatment system of any of claims 27-29, further comprising:
a reservoir for receiving the liquid introduced into the interior space after the flow of gas is exposed to the liquid; and
recirculation element operable to move liquid from reservoir back to the liquid bath.

31. The gas treatment system of any of claims 27-30, further comprising a pump for pumping the liquid from the liquid bath to the liquid introduction element.

32. The gas treatment system of any of claims 27-31, further comprising a filter disposed such that the flow of gas passes through the filter after being exposed to the liquid, the filter being a hydrophilic or hydrophobic filter.

33. The gas treatment system of any of claims 27-32, further comprising a chilled element disposed such that the flow of gas passes through the filter after being exposed to the liquid, thereby removing humidity from the flow of gas.

34. The gas treatment system of any of claims 27-33, further comprising a chilling element operable to reduce a temperature of the liquid before the liquid is introduced into the interior space.

35. The gas treatment system of any of claims 27-34, wherein the flow of gas is a flow of air.

36. The gas treatment system of any of claims 27-35, wherein the system has an outlet for the flow of gas to exit the system after passing through the column.

37. The gas treatment system of claim 36, wherein the outlet is in fluid communication with the discharge gap such that a flow of free radicals is mixed into the flow of gas exiting the system.

38. A system for treatment of an area, comprising the system of claim 37.

39. A system for treatment of an area, comprising:
a free radical generator according to any of claims 3-19; and
a gas flow generating element operable to flow a gas through the discharge gap, thereby introducing free radicals into the flow of gas.

40. A dehumidifier, an air conditioner, or a ventilation system comprising a gas treatment system of any of claims 27-36.

41. A method of fumigating a space or local surface treatment in the space, comprising:
providing the system according to claim 38 or 39;
introducing the flow of gas with free radicals from the system into the space.

42. The method of claim 41, wherein the liquid in the liquid bath contains one or more of: Iodine; Hydrogen Peroxide; Quaternary ammonium; L-Lactic Acid; Peracetic Acid; Citric Acid; Chlorine Dioxide; Isopropanol; Sodium Hypochlorite; or Ethanol.

43. A method of treating a gas, comprising:
providing the generator according to any of claims 1-19 or the system according to any of claims 34-37; and
flowing a gas to be treated through the generator or system.

44. A process of producing a radical in a fluid comprising:
passing a fluid through the discharge gap of the free radical generator of any one of claims 1-19;
applying a pulse voltage between the one or more discharge electrode pins and the counter electrode, the pulse voltage applied for a pulse time; and
generating one or more streamers extending between the one or more discharge electrode pins and the counter electrode, the one or more streamers generating a free radical within the fluid.

45. The process of claim 44, wherein the fluid is a gas.

46. The process of claim 45, wherein said gas comprises oxygen and nitrogen.

47. The process of claim 46, wherein the radical is a nitrogen radical or a hydroxyl radical.

48. The process of claim 44, wherein the pulse voltage is 5 kV to 20 kV.

49. The process of claim 44, further comprising applying a bias voltage between the discharge electrode pins and the counter electrode, the applying being successive voltage pulses.

50. The process of claim 49, wherein the bias voltage is 1 to 500 V or 1 to 2000V.

51. The process of any of claims 44-50, wherein a gas velocity is 0.1 m/s to 200 m/s, optionally 5 m/s to 50 m/s.

52. The process of claim 44, further comprising repeating the step of applying and the step of generating, the repeating defined by a pulse width, the pulse width from 10 nanoseconds to 50 microseconds, optionally 400 nanoseconds to 1 microsecond.

53. The process of claim 44, further comprising repeating the step of applying and the step of generating, the repeating defined by a pulse frequency, the pulse frequency from 100 Hz to 100 kHz, optionally 10 kHz to 30 kHz.

54. The free radical generator of claim 1, wherein the discharge electrode pins each comprise 3 to 8 ignition tips.

55. The free radical generator of claim 1, wherein the discharge electrode pins each comprise a tip profile substantially perpendicular to a length of the discharge electrode pin, wherein the tip profile is in the shape of a triangle, a square, a pentagon, a hexagon, a heptagon, or an octagon.

56. A system for fumigation of crops in an interior grow enclosure, comprising:
an indoor grow enclosure configured to have crops disposed therein; and
an air treatment system according to any of claims 34-37.

57. A method for fumigation of crops in an interior grow enclosure, comprising:
providing an indoor grow enclosure with crops disposed therein;
providing an air treatment system according to any of claims 34-37;
passing air through the system so as to introduce free radicals and/or other reactive species into the air passing through the system; and
introducing the air with the free radicals and/or other reactive species into the indoor enclosure;
thereby exposing the crops to the air with the free radicals and/or other reactive species.

58. A system for sterilizing a device such as a medical instrument, comprising:
a housing having a receiving area for receiving the medical instrument, the receiving area including a liquid bath disposed therein such that the device received in the receiving area is at least partially positioned in the liquid bath;
a discharge electrode assembly comprising a plurality of discharge electrode pins in an array arranged in a radial pattern and electrically configured to receive one or more voltage pulses;
a portion of the liquid bath defining a counter electrode having a surface separated from the discharge electrode pins by a discharge gap; and
a motive element operable to rotate the discharge electrode assembly such that the array of discharge electrode pins rotates about an axis and the plurality of pins move relative to the counter electrode.

59. The system according to claim 58, further comprising a dividing element separating the portion of the liquid bath defining the counter electrode from a portion of the liquid bath receiving the device for sterilization.

60. The system according to claim 58, further comprising an impeller configured for circulating the liquid bath.

61. A method of sterilizing a device such as a medical instrument, comprising:
providing a system according to any of claims 58-60;
receiving the device in the receiving area;
generating active species in the liquid bath with the discharge electrode assembly, thereby generating a sterilant liquid; and
flowing the sterilant liquid over a surface of the device, thereby sterilizing the device.
